(19) United States
(12) Patent Application Publication
(10) Pub. No.: US 2006/0155200 A1
(43) Pub. Date: Jul. 13, 2006

(54) Method for prediction of cardiac disease

(75) Inventor: Leong Loke Ng, Leicester (GB)

Correspondence Address: FOLEY HOAG, LLP;PATENT GROUP, WORLD TRADE CENTER WEST, 155 SEAPORT BLVD, BOSTON, MA, 02110, US

(73) Assignee: University of Leicester, Leicester (GB)

(21) Appl. No.: 10/532,359

(22) PCT Filed: Oct. 21, 2003
(86) PCT No.: PCT/GB03/04541

§ 371 Date: Oct. 31, 2005

(30) Foreign Application Priority Data
Oct. 21, 2002 (GB) 0224425.9

(51) Int. Cl. A61B 5/04

(52) U.S. Cl. 600/509

(57) ABSTRACT

A method for screening patients for heart failure such as left ventricular systolic dysfunction (LVSD) comprises measuring a biomarker and taking an ECG measurement and combining both as factors to obtain a result indicative of the probability of the patient having heart failure. As a result the level of accuracy of the screening is significantly increased. Preferred biomarkers are natriuretic peptides (e.g. ANP, BNP).

### FIELD OF THE INVENTION

This invention relates to the use and measurement of cardiac biomarkers and additional cofactors in the screening of patients for heart failure, for example, left ventricular systolic dysfunction (LVSD). In particular it relates to the use of cardiac biomarkers in combination with electrocardiogram (ECG) and history of ischaemic heart disease and optionally other risk factors indicative of cardiac disease. This invention also relates to an algorithm in order to interrogate the patient's natriuretic peptide level in combination with, in particular major abnormalities found in the patient's ECG data in order to obtain an improved indication of the likelihood of a patient either having or not having LVSD.

### BACKGROUND OF THE INVENTION

Heart failure is a chronic, progressive disease that affects 1.5-2% of the general population of the Western World. Clinically, the term ‘heart failure’ is applied to the syndrome of breathlessness and fatigue, often accompanied by fluid retention, as indicated by an elevated jugular venous pressure and oedema In persons over the age of 65 years, the incidence increases to 6-10%. Heart failure is the most frequent cause of hospitalisation in elderly patients and is recognised as a major health problem. In the USA, 4.6 million individuals have a diagnosis of heart failure and a further 400,000 to 700,000 patients are diagnosed each year, costing the healthcare system nearly $38 billion for in-patient ($23.1 billion) and out-patient care ($14.7 billion) each year. In particular, hospital admission and readmissions account for the majority of this expenditure. Latest findings estimate that as many as 20m people with heart failure in the USA are undiagnosed.

Heart failure is most commonly due to LVSD where the myocardium fails to contract normally and the left ventricle is usually dilated. Previous acute myocardial infarction (AMI), chronic hypertension, dilated cardiomyopathy, viral myocarditis, Chagas' disease and alcoholic heart disease are common causes of myocardial systolic failure.

Patients with heart failure are categorised into different risk groups according to the New York Heart Association (NYHA) functional classification system. This system relates symptoms to everyday activities and the patient's quality of life. The system has four classes. In Class I, patients have cardiac disease but without the resulting limitations of physical activity. Ordinary physical activity does not cause undue fatigue, palpitation, dyspnea, or anginal pain. In Class II, patients have cardiac disease resulting in slight limitation of physical activity. They are comfortable at rest. Ordinary physical activity results in fatigue, palpitation, dyspnea, or anginal pain. In Class III, patients have cardiac disease resulting in marked limitation of physical activity. They are comfortable at rest. Less than ordinary physical activity causes fatigue, palpitation, dyspnea, or anginal pain. Finally, in Class IV, patients have cardiac disease resulting in inability to carry on any physical activity without discomfort. Symptoms of cardiac insufficiency or of the anginal syndrome may be present even at rest. If any physical activity is undertaken, discomfort is increased.

It will be clear that individuals with Class I heart failure and some patients with Class II heart failure cannot easily be identified from patients without heart failure in the general population using clinical history alone. Therefore, in a group of apparently healthy individuals who do hot have any presenting symptoms or obvious recent symptoms of heart failure, identifying these patients using the NYHA criteria for further investigation is not possible. Even if the patient had symptoms suggestive of heart failure, these symptoms overlap with many other conditions and on their own are not specific for heart failure.

Because early stages of heart failure go undetected, diagnosis of heart failure often only occurs when the patient's condition is at a more advanced stage, for example, following presentation to the hospital with acute decompensation. However, it is known that a proportion of apparently healthy individuals will indeed have LVSD. Further, it is known that these patients if treated will benefit from slowed disease progression, fewer hospital re-admissions, and an improved quality of life.

The definitive method to diagnose heart failure is echocardiography. The echocardiogram provides an accurate means to diagnose LVSD and hence heart failure. However, echocardiogram is a skilled technique requiring expertise and is not available to the generalist physician. Further, echocardiography is relatively expensive and access to echocardiography facilities for the generalist physician is frequently inadequate. In routine practice, therefore, generalist physicians rely on clinical features to make a presumptive diagnosis of heart failure, a strategy known to be inaccurate.

Other tests are available to the generalist physician that might have a role in identifying previously undiagnosed patients with LVSD. Studies have evaluated the use of electrocardiography (ECG) and natriuretic peptides.

The resting ECG is the most widely used cardiovascular diagnostic test. Currently, approximately one half of all ECGs are performed by physicians without special training in cardiology. The value of any screening test depends critically on four key principles: its cost; the prevalence of the abnormalities detected in the population assessed; the relationship of the abnormalities to morbidity and mortality; and the possibility of reducing or avoiding future morbidity or mortality given the information provided by the test. In particular, to be worth the additional expense, the ECG must add significantly to the ability of standard risk factors to identify previously undiagnosed individuals with sub-clinical disease. The validity of using the resting 12-lead electrocardiogram as a screening test for cardiovascular disease in apparently healthy individuals has never been convincingly demonstrated.

While some studies have suggested that a significant proportion of patients with LVSD have a normal ECG, others have concluded that the ECG is unlikely to be normal in the patient with LVSD. A significant proportion of patients with LVSD have a normal ECG (Houghton et al: Int. J. Cardiol. 1997; 62: 31-36). Unsurprisingly, previous screening studies have reported a higher prevalence of ECG abnormalities in patients with LVSD than in those with preserved LV systolic function. However, the prevalence of ECG abnormalities in the general population, and in particular in those age groups at risk of LVSD, has been reported to be of the order of 40-75%.

Natriuretic peptides (e.g. atrial natriuretic peptide [ANP], B-type natriuretic peptide [BNP], and their respective prohormones, N-terminal proANP [NTproANP or N-ANP] and N-terminal proBNP [NTproBNP or N-BNP]) have been found to be elevated in patients with LVSD. Studies have focused on the diagnostic and prognostic usefulness of natriuretic peptide measurement. BNP in particular has been demonstrated to discriminate cardiac and non-cardiac dyspnea (i.e. breathlessness), provide prognostic data on future left ventricular function and survival when measured within the days following myocardial infarction, and allow patients with heart failure to be stratified into risk groups dependent on their BNP level.

BNP is a cardiac neurohormone secreted from the cardiac ventricles as a response to ventricular volume expansion and pressure overload. Levels of BNP are elevated in cardiac disease states associated with increased ventricular stretch. BNP levels are reflective of left ventricular diastolic filling pressures and thus correlate with pulmonary capillary wedge pressure. BNP levels have been shown to be elevated in patients with symptomatic left ventricular dysfunction and correlate with New York Heart Association (NYHA) classification and prognosis. Distinguishing-congestive heart failure from other causes of dyspnea is of great importance in patients presenting for medical attention with signs and/or symptoms that may or may not represent heart failure. A number of studies have demonstrated the limited reliability of the physical examination and Chest X-ray in diagnosing heart failure. Even with the best of clinicians, diagnosing heart failure remains a clinical challenge. BNP measurements are now in routine use in the emergency department and urgent-care settings. This assay represents the first clinically available blood test to facilitate the diagnosis of heart failure in patients presenting with symptoms.

BNP can be measured using standard laboratory immunoassay methods (e.g. Shionogi SHIONORIA BNP). Further, a new point-of-care (POC) diagnostic for use in the emergency room or decentralised settings (e.g. heart failure clinic) is now available. The POC assay for BNP, known as the Triage BNP Test, is commercially available from Biosite Incorporated. The assay utilises a fluorescence detection system to measure BNP in whole blood within 15 minutes. The result of the measurement is displayed as a concentration of BNP found in the patient's blood sample, reported in pg/ml. The lower limit of detection is 20 pg/ml and a diagnostic level to exclude heart failure is BNP<100 pg/ml. A level of >100 pg/ml is considered positive and indicative of heart failure.

Studies that have investigated the utility of the natriuretic peptides in population screening for LVSD have observed that the positive predictive value of natriuretic peptides for the presumptive diagnosis of heart failure is low. Thus detection of elevated levels of BNP is not necessarily indicative of the presence or likelihood of LVSD. Patients with lung cancer, pulmonary embolism, myocardial infarction, and end-stage renal disease can also have elevated levels. For this reason, measurement of BNP in a healthy population will identify a significant number of patients with elevated levels, many of whom will not have LVSD. The positive predictive value of the natriuretic peptides in screening unselected populations (i.e. the general population) has been demonstrated to be <20%. These studies have evaluated the predictive value of a natriuretic peptide value above one or more given concentrations. The conclusions made by the authors of these studies is that natriuretic peptide levels have a high negative predictive value (a ‘normal’ value effectively ruling out LVSD), but the positive predictive value is weak (an ‘elevated’ natriuretic peptide value does not necessarily mean that the patient has LVSD). Further, none of the studies have attempted to assess the specificity and positive predictive value of the natriuretic peptides at 100% sensitivity, an important requirement for an effective screening method. In practical terms, if a generalist physician used the results of natriuretic peptide measurements alone at a cut-off optimised to include all patients with LVSD, he will also rule-in a substantial number of patients without LVSD. Whilst it is accepted that measurement of natriuretic peptides is a useful tool in confirming that patients presenting with dyspnea in the acute setting have LVSD, use of natriuretic peptides on their own are of limited use in the identification or screening of patients with LVSD in the community. Similarily, as described above, the use of ECG measurement on its own is also of limited value.

Other biomarkers whose levels are known to be altered in patients with LVSD have been described in the literature. These include Endothelin-1, Big Endothelin-1, Adrenomedullin, Urotensin, Angiotensin II, Uroguanylin, and cell injury markers including troponin I and T. Similar to the natriuretic peptides, consideration of the level of these biomarkers does not enable a clear distinction between patients with or without LVSD.

There is no simple method that would enable a generalist physician to identify a group of patients with previously undiagnosed LVSD without including an unacceptable number of false-positive patients. Similarly, for an individual, there exists a need to be able to identify the existence of LVSD with a reasonable degree of confidence such that that patient is not sent unnecessarily for further investigation.

For the first time it is possible to cost-effectively screen normal apparently healthy individuals to identify patients with a high risk of LVSD. These patients can then be investigated further using echocardiography. The benefit to the patient is that their condition will be identified at an earlier stage (before they present with clinical symptoms) allowing appropriate effective treatment to be implemented. The benefit to society is that patients with heart failure will lead healthier lives for longer and will not consume the same level of resources as they would if they were first identified following hospital admission for, for example, an episode of acute decompensation. There exists therefore a need for an improved means of screening patients in order to identify those with previously undiagnosed LVSD, thus enabling earlier intervention.

### SUMMARY OF THE INVENTION

This invention overcomes the shortcomings of the above-mentioned prior art and provides a method for the screening of patients in order to identify a patient or a group of patients in whom the probability of LVSD is high.

The invention also concerns an algorithm in order to process the data obtained from both the ECG and natriuretic peptide measurements such as to give an indication of the likelihood of a patient having LVSD. Importantly, the use of the algorithm is essential for this result to be obtained. In other words, the same result could not be achieved merely from the consideration of the particular natriuretic peptide concentration in combination with study of the particular ECG traces. The invention as described herein refers to the measurement of a natriuretic peptide. However, it will be obvious that an alternative biomarker such as one of those listed above could be used to construct the algorithm using the methods described.

According to one embodiment, the invention also provides for a device for measuring BNP and/or ECG. The device for measuring natriuretic peptides and/or ECG would either have the algorithm contained within the device by incorporation into software or have the means to receive the result as calculated by the algorithm remotely from the device. Additionally a device to measure either natriuretic peptides or ECG alone would have the means to accept respectively the results obtained from the ECG and natriuretic peptide measurements. Alternatively, the algorithm could reside on a computer and the user input the data obtained from both the natriuretic peptide measurement and the ECG data.

The objectives of the invention have been achieved by consideration of various cofactors in combination with natriuretic peptide measurements. In particular these cofactors relate to data obtained from ECG measurements and considerations of a previous history of myocardial infarction (MI) or angina In particular, these cofactors concern a major ECG abnormality (i.e. Q-wave, left-bundle branch block, left ventricular hypertrophy or atrial fibrillation) and a history of MI or angina Moreover, the predictive value of the model is weakened only minimally by consideration of a natriuretic peptide and the ECG alone. Addition of the ECG result reduces the number of patients who would require an echocardiogram based solely on a natriuretic peptide level by four-fold.

As expected, the measurement of a natriuretic peptide alone, for example BNP, resulted in an unacceptably low positive predictive value of 2.24%. Also, in agreement with previous studies, we found that a high prevalence of ECG abnormalities in the general population (24% had a minor and 16% a major ECG abnormality). Therefore, as a screening tool, ECG suffered from a lack of sensitivity and specificity for the prediction of LVSD.

Interpretation of natriuretic peptide measurement and ECG together increased specificity of the test significantly without any loss of sensitivity (retained at 100%). In terms of screening a low risk population, randomly selected from primary care and without a prior diagnosis of heart failure, all patients with the condition can be identified while minimising the number of patients unnecessarily requiring echocardiographic examination.

We have defined an algorithm employing the patient's natriuretic peptide level alongside additional cofactors, in particular major abnormalities found in the patient's ECG. The algorithm identifies all patients with LVSD and a substantially reduced number of false-positives. This provides for the first time a method that can be used to cost-effectively screen patients for previously undiagnosed heart failure. The improved specificity achievable at 100% sensitivity results in fewer subjects from the population needing to be investigated further by echocardiography scans.

Embodiments of the invention will now be described, by way of example, with reference to the drawings, of which:

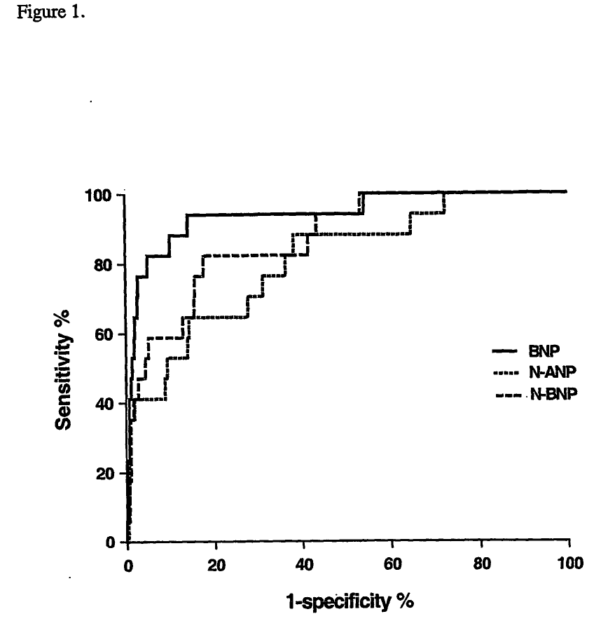

FIG. 1 represents ROC curves for the 3 peptides (N-ANP, BNP and N-BNP) in diagnosis of heart failure (as defined by a LVWMI score≧2;

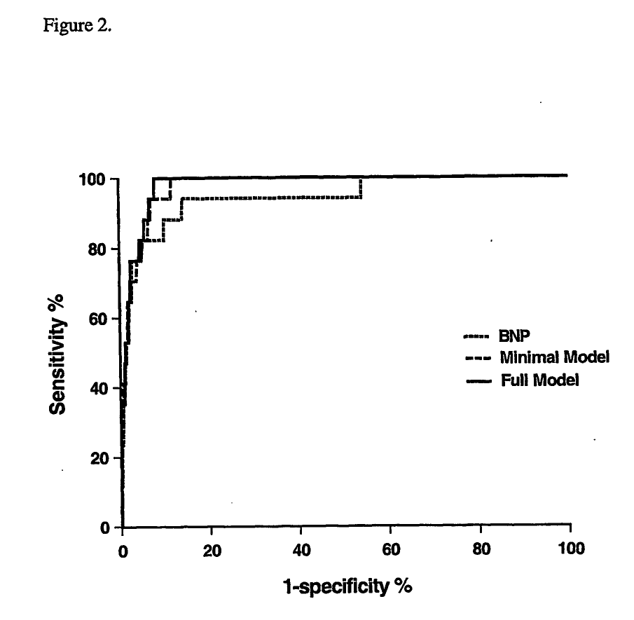

FIG. 2 represents ROC curves for BNP, full model (including log₁₀ BNP, ECG and history of MI or angina) and minimal model (including log₁₀ BNP level and ECG) in diagnosis of heart failure (as defined by a LVWMI score≧2);

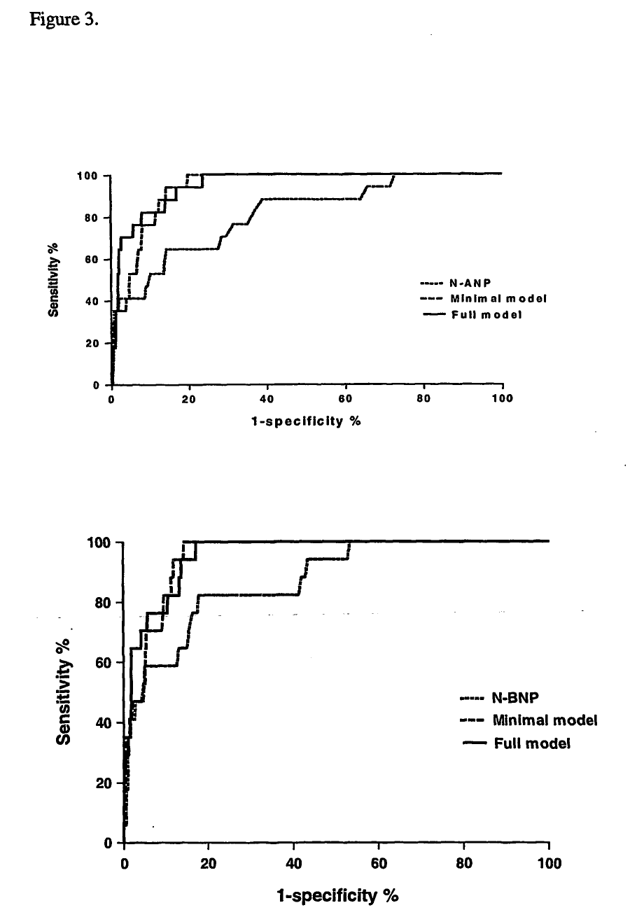

FIG. 3 represents ROC curves for N-ANP and N-BNP, their respective full models (including log₁₀ peptide level, ECG and history of MI or angina) and minimal model (including log₁₀ peptide level and ECG) in diagnosis of heart failure (as defined by a LVWMI score≧2);

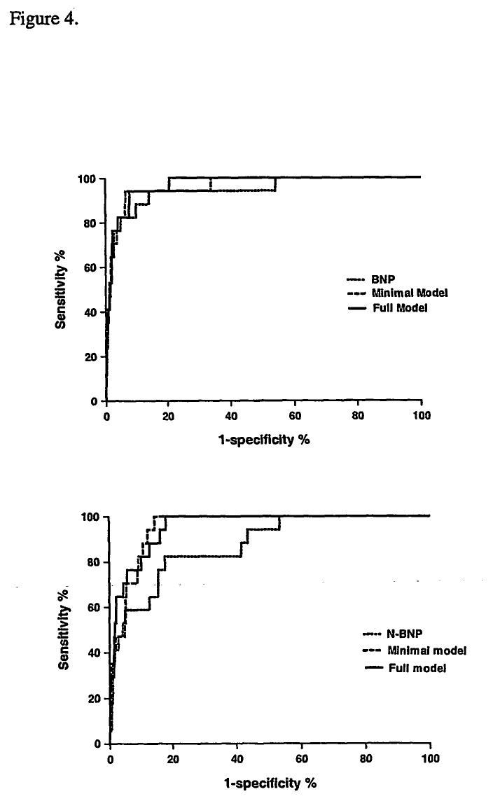

FIG. 4 represents ROC curves for BNP and N-BNP, their respective full models (including peptide level ranked as a percentile, ECG and history of MI or angina) and minimal model (including peptide level ranked as a percentile and ECG) in diagnosis of heart failure (as defined by a LVWMI score≧2); and

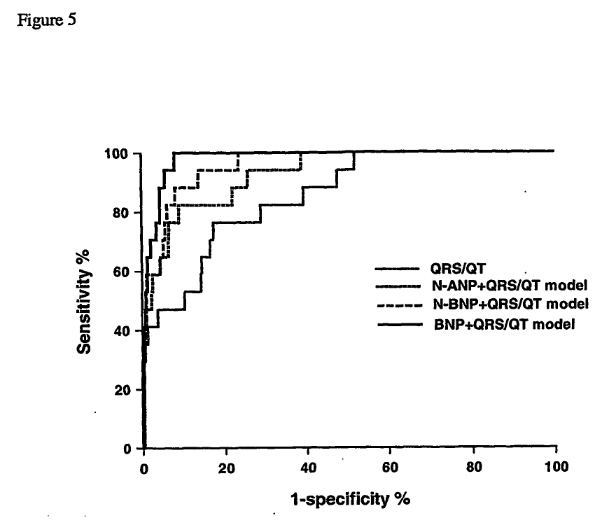

FIG. 5 represents ROC curves for the diagnosis of heart failure as defined by LVMI>2.0. The ROC curves for logistic models with the different peptides combined with log₁₀ QRS/QT ratio are illustrated, together with the ROC curve for the log₁₀ QRS/QT ratio alone.

### DETAILED DESCRIPTION OF THE INVENTION

Thirteen hundred and thirty nine patients (men aged 45-80 and women aged 55-80) of 2392 patients who were approached were entered into the study. All selected patients were from primary care and without a prior diagnosis of heart failure or LVSD. Demographic features are shown in Table 1. Information collected included past medical history of ischaemic heart disease (myocardial infarction or angina), hypertension, diabetes, smoking status, information on prescribed medication, and a check that the patient had not had a confirmed prior diagnosis of heart failure or LVSD. The criterion standard used to diagnose LVSD was echocardiography performed using a Sonos 5500 machine (Philips Technologies). Wall motion score indices (where a score of >2 is indicative of hypokinesis, akinesis, or dyskinesis) and ejection fractions measured during echocardiography were obtained using recognised methods.

In order to measure the levels of natriuretic peptide, twenty mls of peripheral venous blood was drawn into pre-chilled Na-EDTA (1.5 mg/ml blood) tubes containing 500 IU/ml aprotinin. After centrifugation at 3000 rpm at 4° C. for 15 min, plasma was separated and stored at −70° C. until assay. It will be appreciated that any other appropriate bodily fluid sample can be used.

Prior to assay of N-ANP and BNP, plasma was extracted on C₁₈ Sep-Pak (Waters) columns and dried on a centrifugal evaporator. Assays for N-ANP and BNP were based on commercially available antibodies from Peninsular Laboratories Inc (Belmont, Calif., USA) and Phoenix Pharmaceuticals Inc.(Belmont, Calif., USA) respectively. The tracer peptides were biotinylated using biotin-X-N-hydroxysuccinimide ester (Calbiochem, Nottingham, UK) and purified on reverse phase C₁₈ HPLC using an acetonitrile gradient. Plasma extracts and standards were reconstituted with ILMA (immunoluminometric assay) buffer consisting of (in mmol/l) NaH₂PO₄ 1.5, Na₂HPO₄ 8, NaCl 140, EDTA 1 and (in g/l) bovine serum albumin 1, azide 0.1. ELISA plates were coated with 100 ng of anti-rabbit IgG (Sigma Chemical Co., Poole, UK) in 100 μl of 0.1 mol/l sodium bicarbonate buffer, pH 9.6. A competitive immunoluminometric assay was set up by preincubating 50 ng of the anti N-ANP or BNP IgG with standards or samples within the wells. After overnight incubation, 50 μl of the diluted biotinylated N-ANP or BNP peptide tracer (1 μl/ml of the stock solution) was added to the wells. Following another 24 h of incubation at 4° C., wells were washed 3 times. Streptavidin labeled with methyl-acridinium ester was used to detect the tracer bound in the wells. The lower limits of detection of N-ANP and BNP were 3.4 and 2.0 pM respectively. There was no cross reactivity between these assays.

Unextracted plasma was assayed for N-BNP using a non-competitive immunoluminometric assay which was based on the non-competitive N-terminal proBNP assay described by Karl (Development of a novel, N-terminal-proBNP (NT-proBNP) assay with a low detection limit. Scand J Clin Lab Invest Suppl 1999;230:177-181). Rabbit polyclonal antibodies were raised to the N-terminal (amino acids 1-12) and C-terminal (amino acids 65-76) of the human N-terminal proBNP.

IgG from the sera was purified on protein A sepharose columns. The C-terminal directed antibody (0.5 μg in 100 μL for each ELISA plate well) served as the capture antibody. The N-terminal antibody was affinity purified and biotinylatei Aliquots (20 μL) of samples or N-BNP standards were incubated in the C-terminal antibody coated wells with the biotinylated antibody for 24 hours at 4° C. Following washes, streptavidin labeled with methyl-acridinium ester was used to detect bound biotinylated antibody. The lower limit of detection was 5.7 pM of unextracted plasma. There was no cross-reactivity with ANP, N-ANP, BNP or CNP.

In order to obtain ECG measurements, twelve-lead ECGs were analysed for the presence of major (pathological Q wave, left bundle branch block, left ventricular hypertrophy, atrial flutter/fibrillation) and minor (left axis deviation, right bundle branch block, poor R-wave progression, atrial hypertrophy, non-specific ST segment change, sinus bradycardia or tachycardia) abnormality.

Statistical analysis was performed using the SPSS package (version 11.0, SPSS Inc, IL). Natriuretic peptide levels were normalised by log transformation before analysis. Data was investigated using both parametric and non-parametric analysis of variance to identify which factors and covariates relate to LVSD. Logistic regression analysis was performed for predicting the presence of LVSD with factors and covariates which were related to LVSD in univariate analysis and the probability of membership of either group (the prognostic index) saved for plotting of Receiver Operating Characteristic (ROC) curves. This logistic regression analysis can be performed using different statistical software packages (of which SPSS is an example), and yields an equation for predicting the log of the odds ratio (defined as the ratio of the probability of having LVSD to the probability of not having LVSD), the equation having terms such as a constant and coefficients defined as B₁ to Bₙ (n referring to the number of predictor variables in the equation) by which the different predictor variables are multiplied. When these different terms are added together, the loge of the odds ratio can be determined.

The diagnostic accuracy of different computations of variables is compared with a ROC curve. The ROC curve displays the relationship between the sensitivity and specificity of a test at different test cut-off levels. In the case of screening patients for LVSD, the area under the ROC curve indicates how well the test can separate patients with and without LVSD. An ideal test would have an area under the curve of 1.0 meaning that both sensitivity and specificity of the test is 100 per cent. A test that could not distinguish patients with and without LVSD would have an area under the curve of 0.5. ROC curves can be used to compare the effectiveness of the measurement of an individual biomarker to, alternatively, the effectiveness of a combination of variables including, for example, presence or absence of abnormalities in an ECG trace, the result of a measurement of one or more biomarkers, and the presence or absence of a medical history of myocardial infarction or angina. For a combination of variables, it is necessary to determine by computation, a prognostic index from an algorithm that delivers the highest specificity at 100% sensitivity (or the highest achieveable sensitivity) and hence the best ability to identify with confidence a patient with LVSD.

17 individuals (1.3%) had significant LVSD based on the left ventricular wall motion index (LVWMI) score of ≧2 (equivalent of an ejection fraction of <35%, representing severe heart failure that will benefit from institution of therapy such as angiotensin converting enzyme inhibitors and certain beta blockers). Plasma concentration of each natriuretic peptide was higher in those with LVSD than in those with preserved systolic function: median N-ANP (range) 943.4 (288.4-3020) pM vs 385.0 (5.2-4115.4) pM (p<0.0005); BNP 92.9 (19.0-501.2) pM vs 17.1 (2.0-275.4) pM (p<0.0005); N-BNP 301.6 (38.0-1230.3) pM vs 36.3 (5.8-1174.9) pM, (p<0.0005).

Comparison of the areas under the ROC curves for N-ANP (0.810) BNP (0.943), and N-BNP (0.871) showed BNP to be superior in the detection of LVSD (FIG. 1). At 100% sensitivity for the detection of LVWMI≧2, the specificity of N-ANP (287 pM), BNP (19.2 pM) and N-BNP (37.6 pM) was 27%, 47% and 47% respectively.

Data was further analysed to identify the weight to be attached to respective factors by applying the standard statistical analysis method, logistic regression. Logistic regression involves fitting to the data an equation of the form ‘logit(p)=a+b₁x₁+b₂x₂+b₃x₃+ . . . ’, where logit(p)=logₑ(p/(1−p)).

In a logistic regression analysis for univariate determinants of LVWMI≧2, in an optimisation potential factors or covariates included were log₁₀ plasma natriuretic peptide, age, gender, plasma creatinine, major ECG abnormality, minor ECG abnormality, body mass index, and past history of MI or angina, of diabetes or of hypertension. In a multivariate logistic regression analysis (Table 2), plasma BNP was the strongest predictor of LVSD (p<0.0005), the only other factors retaining independent predictive value being major ECG abnormality (p=0.006) and a history of MI or angina (p=0.029).

A decision of whether there are any major abnormalities in the patient's ECG trace leads to a weighting of, for example, of either ‘0’ or ‘1‘. ‘0’ means there were no major abnormalities in the ECG trace and ‘1’ means that an abnormality was detected. The identification of an abnormality, for example, indicative of left ventricular hypertrophy (LVH), left-bundle branch block (LBBB), atrial fibrillation, or the presence of a Q-wave, is easily identified by visual observation of the ECG trace or alternatively, can be determined using appropriate software such as the GE 12SL ECG analysis computer program from GE Medical Systems. This software makes precise measurements of recorded cardiac signals, then provides an interpretation of the ECG waveforms using classic and newly developed ECG interpretation criteria for both rhythm and morphology.

The ROC curves for BNP alone and for the prognostic index derived from the combination of BNP, the ECG and a history of MI or angina are shown in FIG. 2. The area under the ROC curve increased from 0.94 with BNP alone, to 0.978 with the regression model. It is clear that the specificity of the model was markedly improved compared to that of BNP alone (FIG. 2, Table 3). When the model contained only the two strongest predictors, BNP and major ECG abnormality, the area under the curve was 0.974. Once again specificity of this model was clearly superior to that of BNP alone (FIG. 2).

When the regression analysis was repeated for the prediction of LVEF≦35% rather than for LVWMI, very similar results were obtained. There were 16 individuals with LVEF≦35% and the same factors emerged as predictive on multivariate analysis. The odds ratio for a 50% increase in plasma BNP (2.41) was almost identical to that for prediction of LVWMI≧2 (2.43). However the area under the ROC curve and the specificity of the model when sensitivity was set at 100% were slightly less. This is shown in Table 3 demonstrating sensitivity and areas under the ROC curve of BNP, the prognostic indices derived for the full regression model and the minimal regression model, for the prediction of LVWMI score≧2 and for the prediction of LVEF≦35%. The full regression model contains BNP+ECG+history of MI/angina; the minimal regression model contains BNP+ECG. The quoted specificities are for a sensitivity of 100%.

The ECG alone was neither sensitive nor specific for the identification of LVWMI≧2. Of the 17 individuals with LVWMI≧2, the ECG was normal in 2 (12%). Thus the ECG alone could not attain 100% sensitivity for detection of LVSD in our population. The finding of 1 or more minor ECG abnormalities had a specificity of 60.7% and PPV of 3.1% for both LVWMI≧2 and LVEF≦35%.

The coefficients for the full logistic model including additional cofactors for predicting LVWMI≧2 (constant term, B₁, B₂ and B₃) are presented in Table 4. The log₁₀ BNP level is expressed in pM, presence or absence of a major ECG abnormality is coded as 1 or 0 or any other pair of numbers sufficiently separated to impart a different weighting on the associated co-efficient in the presence or absence of the factor, and presence or absence of an ischaemic heart disease history (MI or angina) coded as 1 or 0 or any other pair of numbers sufficiently separated to impart a different weighting on the associated co-efficient in the presence or absence of the factor. The equations for the full model take the general form:
Logₑ p/(1−p)=Constant+B₁*(log₁₀ BNP)+B₂*(ECG abnormality, 1 or 0)+B₃*(history of MI or angina, 1 or 0)
where p is the probability of having heart failure as defined by LVWMI≧2.

The coefficients for the minimal logistic model for predicting LVWMI≧2 (constant term, B₁ and B₂) are presented in Table 5. The equations for the minimal model take the general form:
Logₑ p/(1−p)=Constant+B₁*(log₁₀ BNP)+B₂*(ECG abnormality, 1 or 0)

Logistic regression analysis was performed to predict LVSD as defined by LVWMI≧2 using either N-ANP or N-BNP levels, and factors such as major ECG abnormality and history of MI or angina. These models and the coefficients for the peptide levels, ECG findings or ischaemic history are presented in Table 4 which shows coefficients for the covariates (peptides) and factors for the full model for diagnosis of heart failure, as defined by a LVMI≧2.0. SEMs of the coefficients are in brackets. Areas under the ROC curves were obtained from the predicted probability odds ratios using the model coefficients. Table 5 also shows coefficients for the covariates (peptides) and factors for the minimal model for diagnosis of heart failure, as defined by a LVMI>2.0. SEMs of the coefficients are in brackets. Areas under the ROC curves were obtained from the predicted probability odds ratios using the model coefficients. For both the full model (peptides, ECG, ischaemic history) and the minimal model (peptides, ECG), the ROC areas for N-ANP and N-BNP were markedly improved (Tables 4, 5, FIG. 3). In addition, the specificities for diagnosis of heart failure (at 100% sensitivity) were also greatly improved when compared to use of the peptides alone. This can be seen from Table 6 in which all figures refer to 100% sensitivity for detection of heart failure in the screening population. The specificities, positive predictive values and % of the screenees that need to be investigated by scanning (due to a positive test result) are presented, for diagnosis of heart failure by the peptides (individually) alone, in combination with the ECG major abnormalities and ischaemic heart disease history (full model) or in combination with ECG major abnormalities (minimal model). The full and minimal models employed log₁₀ peptide values in the model.

This resulted in increased positive predictive values for both the full and the minimal model for all the peptides studied. This improved specificity also resulted in less subjects from the population having a “positive” test, hence markedly reducing the number of screenees that need to be investigated further by echocardiography scans (Table 6).

The use of these logistic equations improves markedly the accuracy of all the natriuretic peptides studied (N-ANP, N-BNP and BNP), resulting in increased specificity of detection of heart failure (at 100% sensitivity), increased positive predictive values and hence the number of false positive tests.

The model can be made to be applicable to different centres where normal ranges may differ, by ranking all the peptide levels and expressing them as percentiles. The percentiles are then entered into logistic regression analysis, with presence or absence of major ECG abnormalities and/or presence or absence of history of MI or angina as factors. The models are then as follows:

Full Model:
Logₑ p/(1−p)=Constant+B₁*(peptide centile, as %)+B₂*(ECG abnormality, 1 or 0)+B₃*(history of MI or angina, 1 or 0)
Minimal Model:
Logₑ p/(1−p)=Constant+B₁*(peptide centile, as %)+B₂*(ECG abnormality, 1 or 0)
where p is the probability of having heart failure as defined by LVWMI≧2.

The coefficients for the full logistic models (with peptides, ECG, history of MI or angina) or minimal models (with peptides and ECG) are presented in Table 7 which shows coefficients for the covariates (peptide levels expressed as rank percentile) and factors for the full model for diagnosis of heart failure, as defined by a LVMI>2.0. SEMs of the coefficients are in brackets. Areas under the ROC curves were obtained from the predicted probability odds ratios using the model coefficients. Table 8 also shows coefficients for the covariates (peptide levels expressed as rank percentile) and factors for the minimal model for diagnosis of heart failure, as defined by a LVMI>2.0. SEMs of the coefficients are in brackets. Areas under the ROC curves were obtained from the predicted probability odds ratios using the model coefficients. The resulting ROC curves plotted from the derived prognostic indices of the models all had greater underlying areas than the ROC curves plotted with peptide data alone (Tables 7, 8 and FIG. 4). The specificities of the logistic models in achieving a diagnosis at 100% sensitivity are presented in Table 9 in which all figures refer to 100% sensitivity for detection of heart failure in the screening population. The specificities, positive predictive values and % of the screenees that need to be investigated by scanning (due to a positive test result) are presented, for diagnosis of heart failure by the peptides (individually) alone, in combination with the ECG major abnormalities and ischaemic heart disease history (full model) or in combination with ECG major abnormalities (minimal model). The full and minimal models employed peptide values ranked as percentiles in the model. Both full and minimal models with all peptides had improved specificities compared to use of the peptide data alone in diagnosis of heart failure (Tables 7,8, FIG. 4). Thus, use of the coefficients reported in Tables 7 and 8 allow the calculation of a prognostic index based on the centile of the peptide used, this having similar utility to the absolute level of the peptide.

Surprisingly, it has been found that the QRS, QT and/or JT intervals in the ECG are particularly representative indicators of potential LVSD, if the intervals or average intervals pass a predetermined threshold value.

As is well known, the cardiac cycle as represented on an ECG includes a P-wave, a QRS or R-wave and a T-wave. The P-wave occurs upon de-polarisation of the atria, the QRS wave upon de-polarisation of the ventricles and the T-wave upon re-polarisation of the ventricles. According to the invention the QRS interval, determined from the beginning of the Q-wave to the end of the S-wave and the QT interval measured from the beginning of the Q-wave to the end of the T-wave is particularly relevant. In particular it is found that QRS duration positively identifies heart failure patients and that QT duration is inversely related to it, when the QRS duration has been taken into account using logistic regression analysis. Accordingly, a particularly preferable predictor is the ratio QRS/QT, reflecting the inverse relationship demonstrated by the QT duration. However, it is also possible to use both the QRS and the QT interval (whether corrected for heart rate or not), within the logistic regression analysis with the peptide level, to derive a predicted probability of the odds ratio for presence of heart failure. The JT interval is defined as the interval from the J point (end of the S wave) to the end of the T wave. It is obvious that the JT interval is equal to the QT interval minus the QRS interval. Thus it is also possible here and in the following discussion to use the JT interval instead of the QT interval in deriving a predictor for heart failure. Thus the algorithms could consist of peptide level with QRS and JT intervals, or peptide levels with the QRS/JT ratio.

In the preferred method, ECGs from patients were scanned and converted into picture files. From these files, images of ECGs were analysed. QRS intervals were determined from the beginning of the Q-wave and end of the S-wave in all the leads and the average QRS interval was calculated. QT intervals were measured from the beginning of the Q-wave to the end of the T-wave in all leads, and the average QT interval was calculated. The ratio QRS/QT was then determined for all the patients. If the QRS interval, QRS/JT or QRS/QT exceeds a certain threshold or if QT or JT fall below a certain threshold this is an indicator of heart failure.

The ROC area for the log₁₀ QRS/QT ratio for diagnosis of heart failure (as defined by LVMI>2) was 0.854 (SEM 0.041), as compared to that of using peptides along (N-ANP 0.811 (0.054), N-BNP 0.871 (0.04), BNP 0.942 (0.031)).

The logarithm of the peptide level (BNP, NANP or NBNP) and QRS/QT ratio is then entered into a logistic regression for the diagnosis of heart failure (as defined by a LVMI>2.0) as detailed previously, substituting the log₁₀ QRS/QT ratio for the absence or presence of major ECG abnormalities. This results in models with their various coefficients tabulated in Table 10 in which coefficients for the covariates (log₁₀ peptides and QRT/QT ratio) the logistic model for diagnosis of heart failure, as defined by a LVMI>2.0. SEMs of the coefficients are in brackets. Areas under the ROC curves were obtained from the predicted probability odds ratios using the model coefficients. The ROC curves for the different peptides combined with the log₁₀ QRS/QT ratio are illustrated in FIG. 5, all the areas under the curves for the models are better than the ROC area for the QRS/QT ratio alone. It will be seen that the risk of heart failure increases with QRS/QT such that any appropriate cut off value can be selected, for example from statistical analysis to identify heart failure risk. The models have ROC areas which are also greatly improved compared to the ROC area for diagnosis of heart failure with peptide levels alone. The ROC areas of these models with peptides levels and QRS/QT ratio are comparable to those incorporating presence of major ECG abnormalities with co-factors (called the “minimal model” above) and with co-factors (called the “full model” above) and indeed could be used as an alternative method for determining the diagnosis of heart failure with greater accuracy than the use of peptide levels alone. This approach allows a continuous variable representative of heart function rather than the binary value provided by presence or absence of ECG abnormality. Furthermore it is possible to measure the QRS, QT or JT interval using a two lead ECG which is a significant improvement over the existing 12 lead requirement More leads could be used to improve accuracy yet further.

The coefficients reported in Tables 4,5,7,8 and 10 have been derived for the assay formats and antibodies stated in these examples. Other assays for the analytes mentioned may yield slightly different numerical results, and this will lead to differences in the coefficients stated.

The predictive values, sensitivity and specificity of these models, together with the % of screenees that need to be investigated by echocardiography due to a positive test are reported in Table 11 in which all figures refer to 100% sensitivity for detection of heart failure in the screening population. The specificities, positive predictive values and % of the screenees that need to be investigated by scanning (due to a positive test result) are presented, for diagnosis of heart failure by the peptides (individually) alone, or in combination with the QRS/QT ratio determined from the ECG. The model employed log₁₀ peptide and log₁₀ QRS/QT values. It can be seen that models incorporating the QRS/QT ratio could substantially reduce the % of screenees that need to be investigated, when compared with the use of peptide levels alone or the QRS/QT ratio alone. In addition, the specificities of the models are improved compared to use of peptide or QRS/QT ratio alone, and the positive predictive values of the models are also better than the use of peptide or QRS/QT ratio alone.

Accordingly, it will be seen that the QRS/QT ratio provides incremental predictive value to any of the natriuretic peptides in the diagnosis of LVSD (heart failure). A device such as an electrocardiograph instrument could for example have installed software that could calculate this parameter, and in combination with a natriuretic peptide level, provide an improved diagnostic accuracy for heart failure. Instruments could also be constructed with 2 hand-held electrodes that could measure this QRS/QT ratio and the ratio utilised in the detection of heart failure, allowing simplified and improved apparatus.

Reverting to bio-markers, given the similar diagnostic accuracy of other biomarkers (for example, Endothelin-1, Big Endothelin-1, Adrenomedullin, Urotensin, Angiotensin II, and Uroguanylin) one would expect the logistic model to work for these biomarkers following derivation of new constants B₁, B₂, and B₃. Therefore, one would expect that these biomarkers could also be used to identify patients with a high probability of LVSD following the approach discussed above.

TABLE 1
Characteristics of study population (n = 1339)

| | |
|---|---|
| Men/Women: (number (%)) | 756:583 (56.40/43.6) |
| Age (years): mean (range) | 63 (45-81) |
| Practice Jarman score: mean (range) | +7.1 (−16.0 -to +41.4) |
| BMI: mean ± SD | 26.7 ± 4.4 |
| Systolic blood pressure: mean ± SD | 135 19) |
| Diastolic blood pressure: mean (SD) | 78 ± 12 |
| Current smoker: number (%) | 261 (19.6) |
| Body Mass Index (kg/m²) | 28.5 ± 4.5 |
| Medical History (n (%)) | |
| Myocardial infarction | 33 (2.5) |
| Angina | 92 (6.9) |
| Hypertension | 322 (24) |
| Diabetes mellitus | 63 (4.7) |
| Prescribed therapy | |
| ACE inhibitor/ARA | 117 (8.8) |
| Loop diuretic | 36 (2.7) |
| Other diuretic | 171 (12.8) |
| Beta-blocker | 151 (11.3) |
| Nitrate | 53 (4) |
| Calcium channel blocker | 134 (10) |
| Digoxin | 9 (0.7) |
| Natriuretic peptides (pM: median (range) | |
| N-ANP | 401.1 (5.2-4115.4) |
| BNP | 20.3 (2-507.9) |
| N-BNP | 44.8 (5.7-1230.2) |

Abbreviations:
SD: standard deviation;
BMI: body mass index;
ACE: angiotensin-converting enzyme;
ARA: angiotensin-II receptor antagonist.

TABLE 2
Multivariate analysis for determinants of LVWMI ≧ 2

| Factor | Odds Ratio | p |
|---|---|---|
| Gender (Male) | 1.2 | 0.809 |
| Creatinine | 1 | 0.443 |
| Major ECG abnormality | 9.8 | 0.006 |
| BNP* | 2.4 | <0.0005 |
| History of MI or angina | 3.9 | 0.029 |

*Odds ratio for 50% increase in BNP

TABLE 3

| | Left Ventricular Wall Motion | | Left Ventricular Ejection | |
|---|---|---|---|---|
| | Index Score ≧ 2 | | Fraction ≦ 35% | |
| | AUC | Specificity % | AUC | Specificity % |
| BNP | 0.943 | 47 | 0.943 | 46 |
| BNP + ECG + | 0.978 | 92 | 0.973 | 88 |
| history of | | | | |
| MI/angina | | | | |
| BNP + ECG | 0.974 | 88 | 0.973 | 88 |

TABLE 4

| | | | PMH of | | | |
|---|---|---|---|---|---|---|
| | Log₁₀ | | angina | | | |
| | Peptide | ECG | or | | Nagelkerke | ROC |
| | (pM) B₁ | B₂ | MI B₃ | Constant | r² | area |
| BNP | 4.681 | 2.249 | 1.352 | −13.230 | 0.517 | 0.978 |
| | (0.996) | (0.831) | (0.615) | (1.762) | | (0.007) |
| Odds | 107.919 | 9.48 | 3.864 | | | |
| Ratio | | | | | | |
| N-ANP | 2.951 | 2.936 | 1.785 | −14.533 | 0.393 | 0.951 |
| | (0.954) | (0.788) | (0.539) | (2.751) | | (0.017) |
| Odds | 19.13 | 18.844 | 5.959 | | | |
| Ratio | | | | | | |
| N-BNP | 2.243 | 2.716 | 1.515 | −10.856 | 0.416 | 0.956 |
| | (0.706) | (0.791) | (0.543) | (1.715) | | (0.014) |
| Odds | 9.421 | 15.115 | 4.547 | | | |
| Ratio | | | | | | |

TABLE 5

| | Log₁₀ | | | | |
|---|---|---|---|---|---|
| | Peptide (pM) | ECG | | Nagelkerke | ROC |
| | B₁ | B₂ | Constant | r² | area |
| BNP | 4.996 | 2.525 | −13.561 | 0.492 | 0.974 |
| | (0.946) | (0.806) | (1.724) | | (0.008) |
| Odds Ratio | 147.818 | 12.496 | | | |
| N-ANP | 3.135 | 3.172 | −14.692 | 0.335 | 0.939 |
| | (0.879) | (0.781) | (2.536) | | (0.014) |
| Odds Ratio | 22.98 | 23.854 | | | |
| N-BNP | 2.408 | 2.949 | −10.929 | 0.374 | 0.974 |
| | (0.648) | (0.779) | (1.611) | | (0.008) |
| Odds Ratio | 11.113 | 19.083 | | | |

TABLE 6

| | | Peptide only | Full model | Minimal model |
|---|---|---|---|---|
| BNP | Specificity % | 47 | 92 | 88 |
| | Positive Predictive | 2.4 | 16.2 | 10.8 |
| | value % | | | |
| | % of screenees | 52 | 8 | 12 |
| | necessary to scan | | | |
| N-ANP | Specificity % | 27 | 76 | 80 |
| | Positive Predictive | 1.8 | 5.4 | 6.5 |
| | value % | | | |
| | % of screenees | 72 | 24 | 20 |
| | necessary to scan | | | |
| N-BNP | Specificity % | 47 | 82 | 85 |
| | Positive Predictive | 2.4 | 7.2 | 8.6 |
| | value % | | | |
| | % of screenees | 52 | 18 | 15 |
| | necessary to scan | | | |

TABLE 7

| | Peptide | | | | | |
|---|---|---|---|---|---|---|
| | ranked | | | | | |
| | as | | PMH of | | | |
| | centile | | angina | | Nagelkerke | ROC |
| | % | ECG | or MI | Constant | r² | area |
| BNP | 0.101 | 2.44 | 1.299 | −14.015 | 0.478 | 0.969 |
| | (0.035) | (0.804) | (0.563) | (3.153) | | (0.013) |
| Odds | 1.106 | 11.467 | 3.665 | | | |
| Ratio | | | | | | |
| N-ANP | 0.026 | 3.121 | 1.745 | −8.177 | 0.367 | 0.949 |
| | (0.012) | (0.777) | (0.537) | (1.099) | | (0.017) |
| Odds | 1.027 | 22.676 | 5.724 | | | |
| Ratio | | | | | | |
| N-BNP | 0.044 | 2.878 | 1.482 | −9.394 | 0.392 | 0.954 |
| | (0.017) | (0.784) | (0.545) | (1.482) | | (0.014) |
| Odds | 1.045 | 17.774 | 4.400 | | | |
| Ratio | | | | | | |

TABLE 8

| | Log₁₀ | | | Nagelkerke | ROC |
|---|---|---|---|---|---|
| | Peptide (pM) | ECG | Constant | r² | area |
| BNP | 0.114 | 2.647 | −14.913 | 0.45 | 0.962 |
| | (0.036) | (0.788) | (3.261) | | (0.019) |
| Odds Ratio | 1.12 | 14.11 | | | |
| N-ANP | 0.032 | 3.324 | −8.251 | 0.311 | 0.938 |
| | (0.012) | (0.77) | (1.095) | | (0.015) |
| Odds Ratio | 1.033 | 27.783 | | | |
| N-BNP | 0.053 | 3.067 | −9.749 | 0.352 | 0.952 |
| | (0.017) | (0.774) | (1.50) | | (0.012) |
| Odds Ratio | 11.113 | 19.083 | | | |

TABLE 9

| | | Peptide only | Full model | Minimal model |
|---|---|---|---|---|
| BNP | Specificity % | 47 | 79 | 66 |
| | Positive Predictive | 2.4 | 6.2 | 3.8 |
| | value % | | | |
| | % of screenees | 52 | 21 | 34 |
| | necessary to scan | | | |
| N-ANP | Specificity % | 27 | 76 | 80 |
| | Positive Predictive | 1.8 | 5.4 | 6.5 |
| | value % | | | |
| | % of screenees | 72 | 24 | 20 |
| | necessary to scan | | | |
| N-BNP | Specificity % | 47 | 82 | 86 |
| | Positive Predictive | 2.4 | 7.2 | 9.2 |
| | value % | | | |
| | % of screenees | 52 | 18 | 14 |
| | necessary to scan | | | |

TABLE 10

| | Log₁₀ | Log₁₀ | | | |
|---|---|---|---|---|---|
| | Peptide | QRS/QT | | | |
| | (pM) | ratio | | Nagelkerke | ROC |
| | B₁ | B₂ | Constant | r² | area |
| BNP | 5.33 | 19.358 | −1.576 | 0.522 | 0.98 |
| | (0.905) | (4.636) | (3.06) | | (0.006) |
| Odds | 206.42 | 2.6 × 10⁸ | | | |
| Ratio | | | | | |
| N-ANP | 4.358 | 24.126 | −2.65 | 0.387 | 0.929 |
| | (0.875) | (4.293) | (3.213) | | (0.027) |
| Odds | 78.065 | 3.0 × 10¹⁰ | | | |
| Ratio | | | | | |
| N-BNP | 3.23 | 24.921 | 3.125 | 0.452 | 0.958 |
| | (0.679) | (4.516) | (2.675) | | (0.015) |
| Odds | 25.29 | 6.7 × 10¹⁰ | | | |
| Ratio | | | | | |

TABLE 11

| | | | Log₁₀ | Model with |
|---|---|---|---|---|
| | | | QRS/QT | peptide and |
| | | Peptide only | alone | QRS/QT |
| BNP | Specificity % | 47 | - | 92 |
| | Positive Predictive | 2.4 | - | 16.0 |
| | value % | | | |
| | % of screenees | 52 | - | 9 |
| | necessary to scan | | | |
| N-ANP | Specificity % | 27 | - | 61 |
| | Positive Predictive | 1.8 | - | 3.3 |
| | value % | | | |
| | % of screenees | 72 | - | 40 |
| | necessary to scan | | | |
| N-BNP | Specificity % | 47 | - | 77 |
| | Positive Predictive | 2.4 | - | 5.5 |
| | value % | | | |
| | % of screenees | 52 | - | 24 |
| | necessary to scan | | | |
| Log₁₀ | Specificity % | - | 48 | - |
| QRS/QT | | | | |
| | Positive Predictive | - | 2.5 | - |
| | value % | | | |
| | % of screenees | - | 53 | - |
| | necessary to scan | | | |

1. A method for screening an individual or group of patients for the likelihood of having left ventricular systolic dysfunction (LVSD) comprising, in any order the steps of:
(a) measurement of the levels of a biomarker in a sample or samples of bodily fluid of said patient; and
(b) conducting an electrocardiography (ECG) measurement on said patient or group of individuals;
identification of the presence or absence of one or more major abnormality factors from the ECG trace;
assigning or calculating weighting factors for (a) and (b); and
obtaining a result indicative of the probability of said individual having LVSD.

2. The method of claim 1, comprising the further step performed in any order in relation to the steps of claim 1 of identification of the presence or absence of one or more cofactors which are known to be risk factors for CVD; and assigning or calculating a weighing factor (c) to obtain said result.

3. The method of claim 1, wherein the weighting factors for (a), (b) and/or (c) are derived by logistic regression analysis on measurements of a biomarker, ECG findings, and of one or more cofactors which are known to be risk factors for CVD; wherein the patient population is taken from the general population and individuals have no previous diagnosis of LVSD.

4. The method of claim 1, wherein the biomarker is a natriuretic peptide.

5. The method of claim 2, wherein one or more cofactors are selected from myocardial infarction (MI) and angina.

6. An algorithm for the determination of the likelihood of an individual of having left ventricular systolic dysfunction (LVSD) according to the following formula:
Logₑ p/(1−p)=Constant+B₁*(y)+B₂*(ECG abnormality, a)+B₃*(history of MI or angina, a)
where p is the probability of having heart failure as defined by LVSD;
B₁, B₂, and B₃ are the coefficients for the logistic model for predicting LVSD;
wherein ‘a’ is a factor to indicate the presence or absence of ECG abnormality and history of myocardial infarction (MI) or angina and wherein ‘a’ refers to any two numbers sufficiently separated as to impart a different weighting on the coefficients B₂ and B₃ in the presence or absence of ECG abnormality and history of MI or angina;
‘y’ is either log₁₀ natriuretic peptide expressed in pM, or peptide centile;
wherein peptide centile, expressed as per cent, is determined by ranking all biomarker levels determined by measuring the biomarker level for an apparently healthy population using a chosen assay kit and expressing them as percentiles.

7. An algorithm for the determination of the likelihood of an individual of having left ventricular systolic dysfunction (LVSD) according to the following formula:
Logₑ p/(1−p)=Constant+B₁*(y)+B₂*(ECG abnormality, a)
where p is the probability of having heart failure due to LVSD
B₁ and B₂ are the coefficients for the logistic model for predicting LVSD;
wherein ‘a’ is a factor to indicate the presence or absence of ECG abnormality and wherein ‘a’ refers to any two numbers sufficiently separated as to impart a different weighting on the coefficient B₂ in the presence or absence of ECG abnormality;
‘y’ is either log₁₀ natriuretic peptide expressed in pM, or peptide centile;
wherein peptide centile, expressed as per cent, is determined by ranking all biomarker levels determined by measuring the biomarker level for an apparently healthy population using a chosen assay kit and expressing them as percentiles.

8. The method of claim 1, in which the identification of the presence or absence of one or more major abnormality factors from the ECG trace is determined from the QRS, QT, and/or JT interval.

9. The method of claim 8, in which the identification of the major abnormality factor is determined from the ratio QRS interval/QT interval or QRS interval/JT interval.

10. A method of deriving an indicator of heart failure in a patient comprising:
measuring as a first factor the level of a cardiac bio-marker in a sample of bodily fluid of said patient;
obtaining a patient electrocardiography (ECG) trace;
identifying as a second factor the presence of absence of one or more abnormality factors from the ECG trace; and
deriving an indicator of heart failure as a function of the first and second factors.

11. The method of claim 10, wherein the cardiac bio-marker is a marker indicative of the presence or absence of heart failure.

12. The method of claim 11, in which the marker is a natriuretic peptide.

13. The method of claim 12, in which the natriuretic peptide is BNP.

14. The method of claim 10, for deriving an indicator of LVSD.

15. A method of deriving an indicator of heart failure in a patient comprising:
obtaining a patient electrocardiography (ECG);
measuring at least one of the QRS, QT and JT interval from the ECG and deriving the indicator of heart failure from the the QRS, JT and/or QT interval.

16. The method of claim 15, in which the indicator is derived as a function of the ratio QRS interval/QT interval or QRS interval/JT interval.

17. The method claim 15, further comprising measuring the level of a bio-marker in a sample of bodily fluid of a patient and deriving the indicator as a function in addition of the measured level.

18. An apparatus for measuring an indicator of heart failure in a patient comprising at least one of a QRS interval detector, a QT interval detector and a JT interval detector.

19. A heart failure indicator apparatus comprising a data processor arranged to receive data representative of the measurement of a level of a bio-marker in a sample of bodily fluid of a patient and data representing an electrocardiography (ECG) measurement on the patient and/or data representing a measurement of at least one of a QRS or a QT or a JT interval in an ECG, the processor being further arranged to process the received data to derive an indicator of heart failure.

20. A kit of parts comprising at least one of a detector for detecting as a factor levels of a bio-marker in a sample of bodily fluid of a patient, a detector for obtaining an electrocardiography (ECG) trace from on a patient, a processor for identifying as a factor the presence or absence of one or more major abnormality factors from the ECG trace; a processor for measuring as a factor at least one of the QRS, QT and JT interval from an ECG trace and a processor for processing measurements to derive an indicator of heart failure as a function of one or more of the factors.

21. A computer program comprising a set of instructions configured to implement a method as claimed in claim 1.

22. A computer configured to implement a computer program as claimed in claim 21.

23. A computer readable medium storing a computer program as claimed in claim 21.
